(19) United States
(12) Patent Application Publication
(10) Pub. No.: US 2016/0374592 A1
(43) Pub. Date: Dec. 29, 2016

(54) RESPIRATORY MONITORING SYSTEM AND RESPIRATORY MONITORING METHOD

(71) Applicant: CHUNGBUK NATIONAL UNIVERSITY INDUSTRY ACADEMIC COOPERATION FOUNDATION, Chungcheongbuk-do (KR)

(72) Inventors: In Kwang LEE, Chungcheongbuk-do (KR); Mi Jung PARK, Chungcheongbuk-do (KR); Kyung Ok KIM, Seoul (KR); Eun Jong CHA, Chungcheongbuk-do (KR); Kyung Ah KIM, Chungcheongbuk-do (KR)

(73) Assignee: CHUNGBUK NATIONAL UNIVERSITY INDUSTRY ACADEMIC COOPERATION FOUNDATION

(21) Appl. No.: 15/174,355

(22) Filed: Jun. 6, 2016

(30) Foreign Application Priority Data
Jun. 25, 2015 (KR) 10-2015-0090518

(51) Int. Cl. A61B 5/091; A61B 5/00; A61B 5/087; A61M 16/00; A61B 5/08; A61B 5/03; A61M 16/08; A61M 16/04

(52) U.S. Cl. CPC A61B 5/091; A61M 16/0875; A61B 5/7278; A61M 16/04; A61M 16/0078; A61B 5/082; A61B 5/03; A61B 5/742; A61B 5/087; A61M 2016/0027; A61M 2230/432; A61M 2205/75

(57) ABSTRACT

A respiratory monitoring system includes: a first sensing tube provided in a respiratory flow tube and provided with at least a first directional hole opened in a respiratory flow direction; a second sensing tube provided with at least a second directional hole corresponding to the first directional hole; a first sensing element configured to detect a first dynamic pressure (P_{L}) using a differential pressure between gas flows from the first and second sensing tubes; a second sensing element configured to detect a second dynamic pressure (P_{H}) using a differential pressure between gas flows from the first and second sensing tubes; and a computation unit configured to compute patient's respiration information including a tidal inspiratory volume and a tidal expiratory volume using the first and second dynamic pressures.

### CROSS REFERENCES TO RELATED APPLICATIONS

The present invention contains subject matter related to Korean Patent Application No. 2015-0090518, filed in the Korean Patent Office on Jun. 25, 2015, the entire contents of which are incorporated herein by reference.

### FIELD

The present disclosure relates to a respiratory monitoring technology, and more particularly, to a system and method for monitoring respiration of a critical patient on a breathing machine basis.

### BACKGROUND

In general, an urgent critical patient is classified on a disease type basis and is treated in an intensive care unit. In every type of the intensive care unit, a respiratory treatment is regarded as an indispensable important treatment process.

The respiratory treatment for a critical patient is necessarily performed on a pathophysiological basis for a disease that causes an acute respiratory failure. For this purpose, an arterial blood gas analysis, a pulmonary compliance check, and the like are applied.

Since a critical patient usually suffers from weak spontaneous breathing or has a coma, it is necessary to forcibly induce breathing. Accordingly, it is very important to continuously monitor a respiratory flow and a respiratory signal of a critical patient and accurately check a respiratory status.

In the case of a patient in cardiac arrest, if artificial cardiopulmonary resuscitation is taken as soon as cardiopulmonary arrest occurs, a survival rate can increase two or three times. Therefore, an initial emergency treatment determines convalescence.

In general, it takes at least 10 minutes or longer until a rescuer arrives at an accident site depending on an emergency status. If a long time elapses after ventricular fibrillation, or a first-aid patient suffers from suffocation cardiac arrest (asphyxial arrest), the patient may already have hypoxia. Therefore, it is more important to apply an artificial respiration amount and an artificial respiration method suitable for a patient's status or condition.

In the respiratory flow measurement techniques known in the art, a flow detection element is positioned in the middle of a flow path, and the gas flow is converted into a pressure or other types of physically measurable variables.

However, since a critical patient spits secretions such as saliva or bloody phlegm from time to time, the flow detection element may be polluted by a high humidity of the measurement gas or foreign substances, and this may affect a measurement characteristic. In addition, since the flow detection element also acts as a resistor to the gas flow, it may also obstruct respiration of a critical patient having a very slow respiration rate of 500 mL/sec or lower.

In order to prevent such an accident, as illustrated in FIG. 1A, in a respiratory monitoring device (pneumotachometer) of the prior art, a respiratory gas flow is computed by arranging a fluid resistor as a flow detection element having a fine mesh or an array of capillaries in parallel with a gas flow path, and a differential pressure is measured between both ends of the fluid resistor while the gas flow passes therethrough.

However, in the pneumotachometer of the prior art, the flow is obstructed by foreign substances such as moisture or secretions accumulated in the fluid resistor, and this generates an unstable measurement result. In addition, since the fluid resistor naturally hinders a respiratory flow, the pneumotachometer of the prior art is not suitable for a critical patient who has weak respirations and is usually employed for a one-time lung function test.

As illustrated in FIG. 2B, in the pneumotachometer of the prior art, a respiratory flow rotates a turbine or a propeller in the middle of the gas flow path, and the rotation number thereof is measured to compute the respiratory flow rate.

However, the pneumotachometer of the prior art has poor dynamic characteristics and is not allowed to perform bidirectional respiration measurement. Furthermore, accumulation of secretions or saliva in a rotational shaft obstructs rotation of the turbine and degrades accuracy in the flow measurement. Therefore, it is difficult to use the pneumotachometer of the prior art for a critical patient who discharges an amount of secretions.

As illustrated in FIG. 3C, in another respiratory monitoring device (hot-wire anemometer) of the prior art, heat energy lost by a gas flow passing through a hot wire is measured on the basis of a temperature change to compute the respiration.

However, in the hot-wire anemometer of the prior art, it is necessary to maintain a constant temperature while an electric current flows as much as the lost heat energy. Therefore, a device structure becomes complicated and has a large size. In addition, since it sensitively responds to secretions or saliva, it is necessary to additionally install a filter or a heater. Therefore, the hot-wire anemometer of the prior art is employed in a certain expensive flow sensor model.

### SUMMARY

In view of the aforementioned problems, it is an object of the present invention to provide a critical patient respiratory monitoring system and a method of monitoring respiration information of a patient in a breathing machine.

According to an aspect of the present invention, there is provided a respiratory monitoring system including: a first sensing tube provided in a respiratory flow tube serving as a flow passage of a breathing machine and provided with at least a first directional hole opened in a respiratory flow direction; a second sensing tube provided with at least a second directional hole corresponding to the first directional hole and provided in the vicinity of the first sensing tube; a first sensing element configured to detect a first dynamic pressure (P_{L}) using a differential pressure between gas flows from the first and second sensing tubes; a second sensing element configured to detect a second dynamic pressure (P_{H}) using a differential pressure between gas flows from the first and second sensing tubes, the second sensing element having sensitivity lower than that of the first sensing element and a sensing range wider than that of the first sensing element; and a computation unit configured to compute patient's respiration information including a tidal inspiratory volume and a tidal expiratory volume using the first and second dynamic pressures, wherein the computation unit computes the respiration information using a lower flow rate (F_{L}) if the lower flow rate (F_{L}) computed from the first dynamic pressure is smaller than a preset threshold value, and the computation unit computes the respiration information using a higher flow rate (F_{H}) computed from the second dynamic pressure if the lower flow rate (F_{L}) is greater than the threshold value.

According to another aspect of the present invention, there is provided a respiratory monitoring method using a respiratory monitoring system having a first sensing tube provided in a respiratory flow tube serving as a flow passage of a manual breathing machine and provided with at least a first directional hole opened in a respiratory flow direction, a second sensing tube provided with a second directional hole corresponding to the first direction hole and provided in the vicinity of the first sensing tube, a first sensing element configured to detect a first dynamic pressure (P_{L}) using a differential pressure between gas flows from the first and second sensing tubes, a second sensing element configured to detect a second dynamic pressure (P_{H}) using a differential pressure between gas flows from the first and second sensing tubes, the second sensing element having sensitivity lower than that of the first sensing element and a sensing range wider than that of the first sensing element, and a computation unit, the respiratory monitoring method including: computing patient's respiration information including a tidal inspiratory volume and a tidal expiratory volume using the first and second dynamic pressures, wherein the respiration information is computed using a lower flow rate (F_{L}) if the lower flow rate (F_{L}) computed from the first dynamic pressure is smaller than a preset threshold value, and the respiration information is computed using a higher flow rate (F_{H}) computed from the second dynamic pressure if the lower flow rate (F_{L}) is greater than the threshold value.

According to the present invention, it is possible to provide respiratory information under various respiratory conditions of a critical patient using a breathing machine.

### BRIEF DESCRIPTION OF THE DRAWINGS

The foregoing and additional features and characteristics of this disclosure will become more apparent from the following detailed description considered with the reference to the accompanying drawings, wherein:

FIGS. 1A, 1B and 1C are diagrams illustrating respiratory monitoring systems of the prior art;

FIG. 1D is a diagram for describing a principle of bidirectional flow measurement according to an embodiment of the invention;

FIGS. 2A, 2B, 2C, 2D and 2E are diagrams illustrating a respiratory monitoring system according to an embodiment of the invention;

FIG. 2F is a diagram illustrating a zero-point correction unit according to an embodiment of the invention;

FIG. 2G is a graph illustrating a respiratory signal according to an embodiment of the invention;

FIGS. 2H and 2I are diagrams illustrating a display unit according to an embodiment of the invention;

FIG. 2J is a diagram illustrating another exemplary structure of first and second sensing tubes according to an embodiment of the invention;

FIG. 2K is a photograph of the respiratory monitoring system according to an embodiment of the invention;

FIG. 3A is a flowchart illustrating a respiratory monitoring method according to an embodiment of the invention;

FIGS. 3B and 3C are graphs illustrating respiratory signals according to an embodiment of the invention;

FIG. 4A is a graph illustrating a correlation between a first dynamic pressure and a flow rate according to an embodiment of the invention;

FIG. 4B is a graph illustrating a correlation between a second dynamic pressure and a flow rate according to an embodiment of the invention; and

FIG. 4C is a graph obtained by using an exemplary characteristic expression of the respiratory signal according to an embodiment of the invention.

### DETAILED DESCRIPTION

Hereinafter, preferred embodiments of the invention will be described in detail with reference to the accompanying drawings. It is noted that like reference numerals denote like elements throughout overall drawings. In addition, descriptions of well-known apparatus and methods may be omitted so as to not obscure the description of the representative embodiments, and such methods and apparatus are clearly within the scope and spirit of the present disclosure. The terminology used herein is only for the purpose of describing particular embodiments and is not intended to limit the invention. As used herein, the singular forms “a,” “an,” and “the” may be intended to include the plural forms as well, unless the context clearly indicates otherwise. It is further to be noted that, as used herein, the terms “comprises,” “comprising,” “include,” and “including” indicate the presence of stated features, integers, steps, operations, units, and/or components, but do not preclude the presence or addition of one or more other features, integers, steps, operations, units, and/or components, and/or combination thereof.

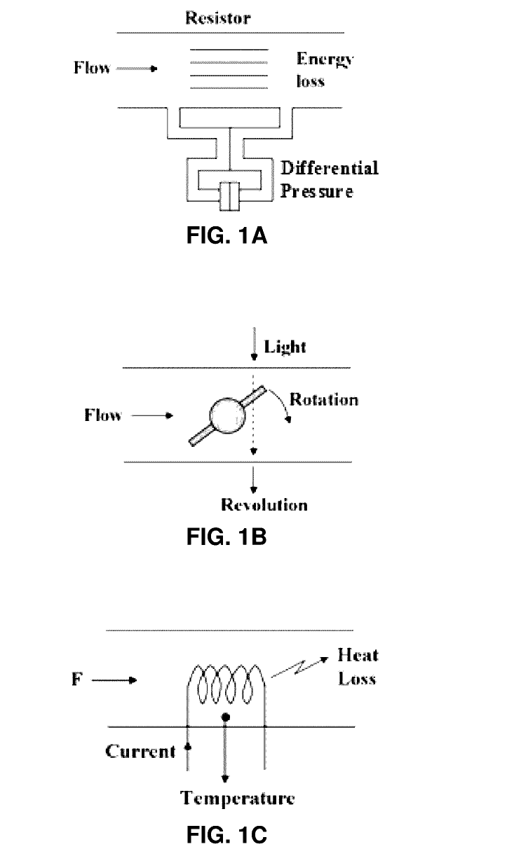

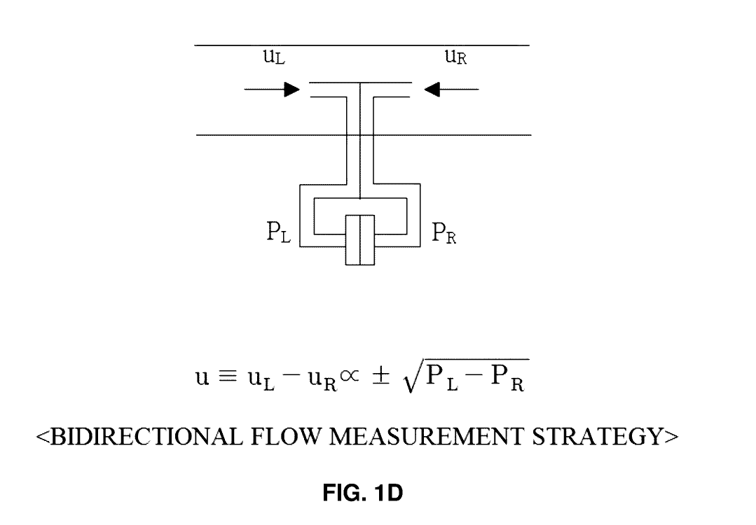

Before description of specific examples of the present invention, a principle of computing inspiratory and expiratory volumes using a pitot tube will be described with reference to FIG. 1D.

Referring to FIG. 1D, a pitot tube as a small-diameter cylindrical flow sensing tube is positioned in parallel with a gas flow, and pressure sensors are connected to measure a pressure P. The pressure P includes a static pressure P_{S} intrinsic to the gas flow and a dynamic pressure P_{D} generated by a motion of the gas flow (P=P_{D}+P_{S}). A differential pressure P_{diff} (=P_{L}−P_{R}) between a pair of pressure sensors of the pitot tube arranged in symmetry is measured only for the dynamic pressure P_{D} relating to a flow velocity u because the static pressure P_{S} is compensated when there is no energy loss between two positions. Here, “u_{L}” denotes a flow velocity when the gas flows from the left to the right, and “P_{L}” denotes a pressure when the gas flows from the left to the right. Similarly, “u_{R}” and “P_{R}” denote a flow velocity and a pressure, respectively, when the gas flows from the right to the left. In addition, the reference signs of the dynamic pressure P_{D} can also be represented by using the left or right flow direction (for example, a respiration flow can be classified into expiratory and inspiratory flows).

The respiratory flow can be expressed by a time-dependent rate of change of the volume of the moving gas. Therefore, assuming that the cross-sectional area A of the gas flow tube is constant, the flow velocity u is proportional to a respiratory flow rate F. Accordingly, the respiratory flow rate F can be obtained by measuring the dynamic pressure P_{D}.

$$F = \frac{d V}{d t} = A \cdot u \qquad [ Formula 1 ]$$

By integrating the respiratory flow rate F using the following Formula 2, it is possible to obtain patient's tidal inspiratory and expiratory volumes. Based on this principle, according to the present invention, a patient's tidal inspiratory volume [mL] is computed by integrating the respiratory flow rate during an inspiratory period, and a patient's tidal expiratory volume [mL] is computed by integrating the respiratory flow rate during an expiratory period.

V(t)=∫F(t)dt [mL] [Formula 2]

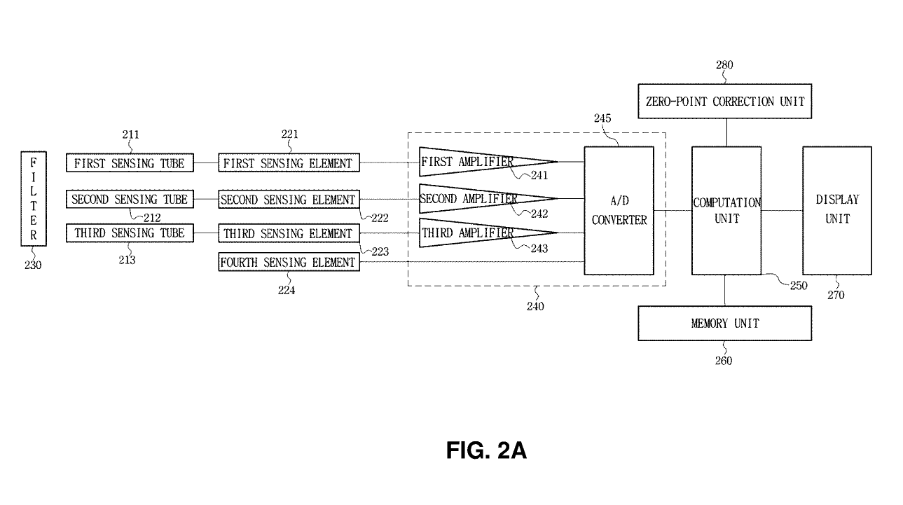

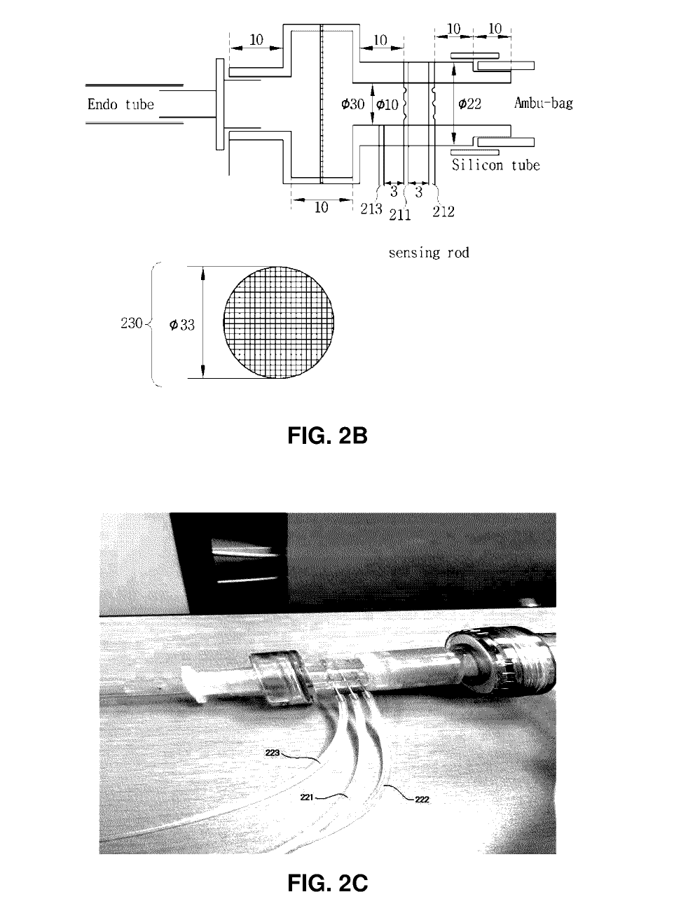

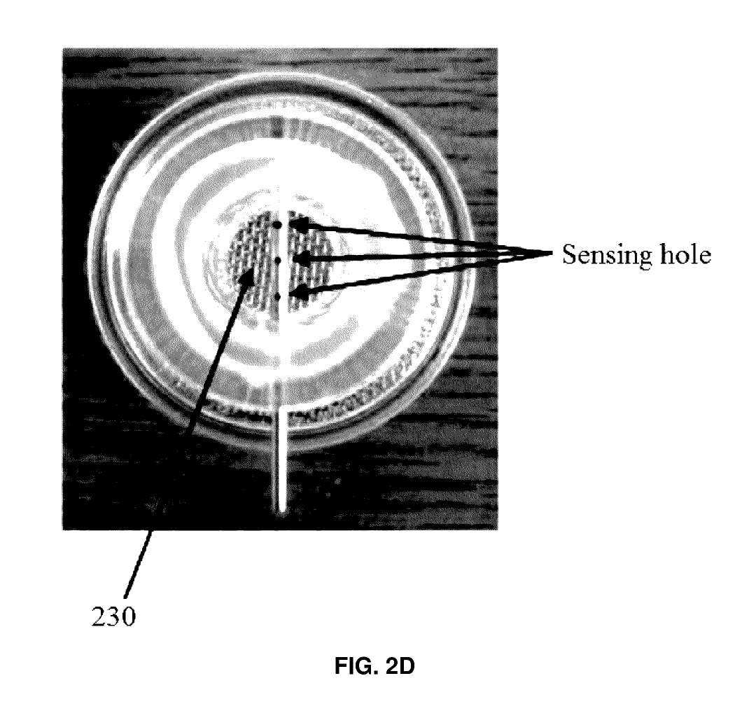

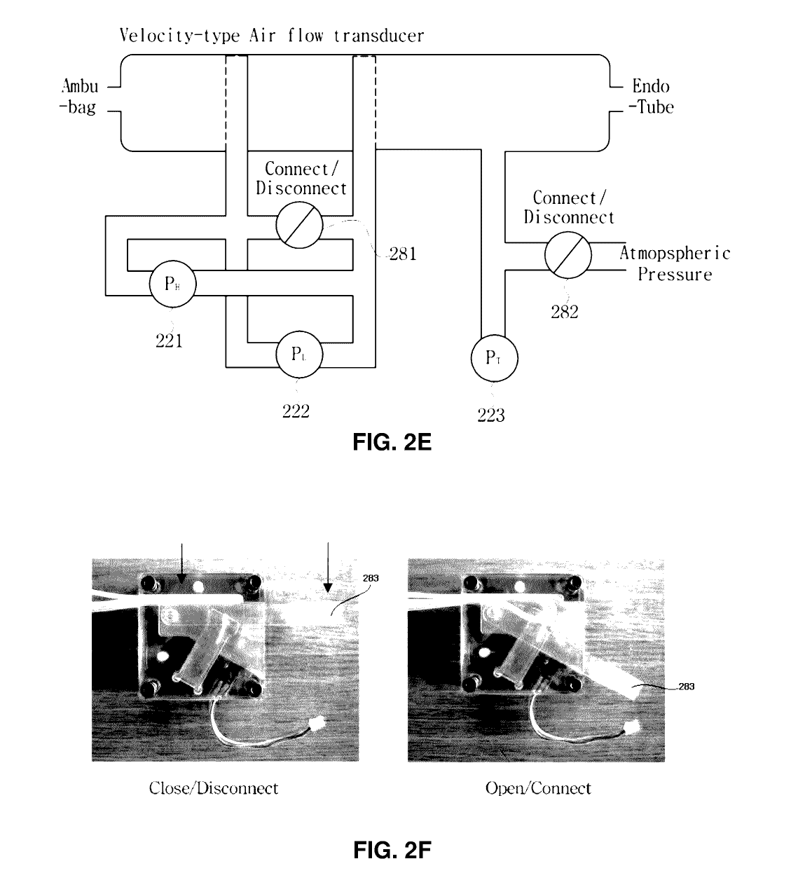

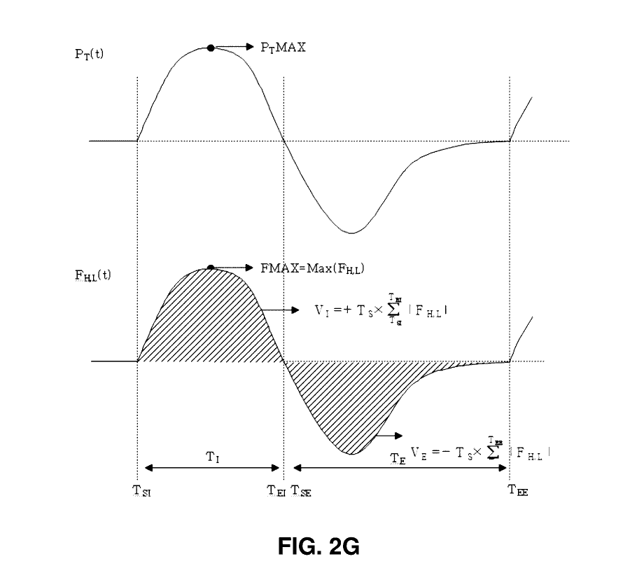

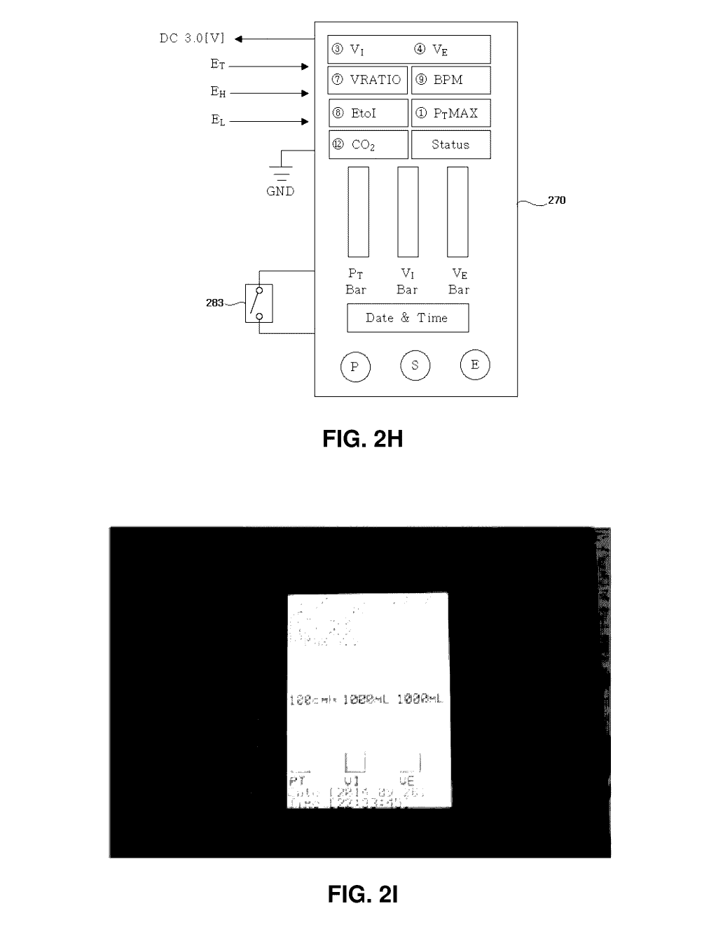

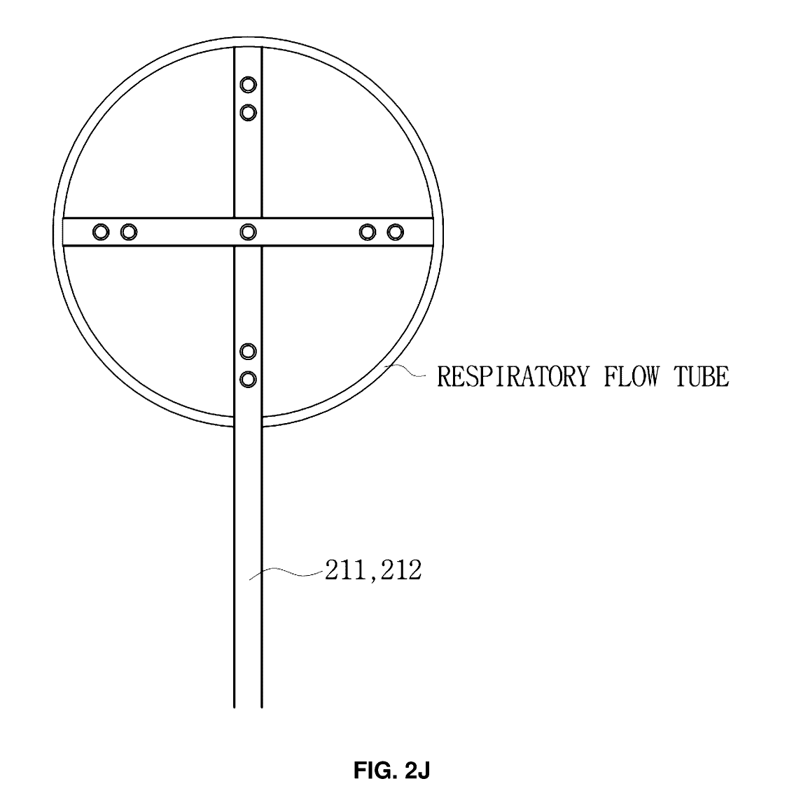

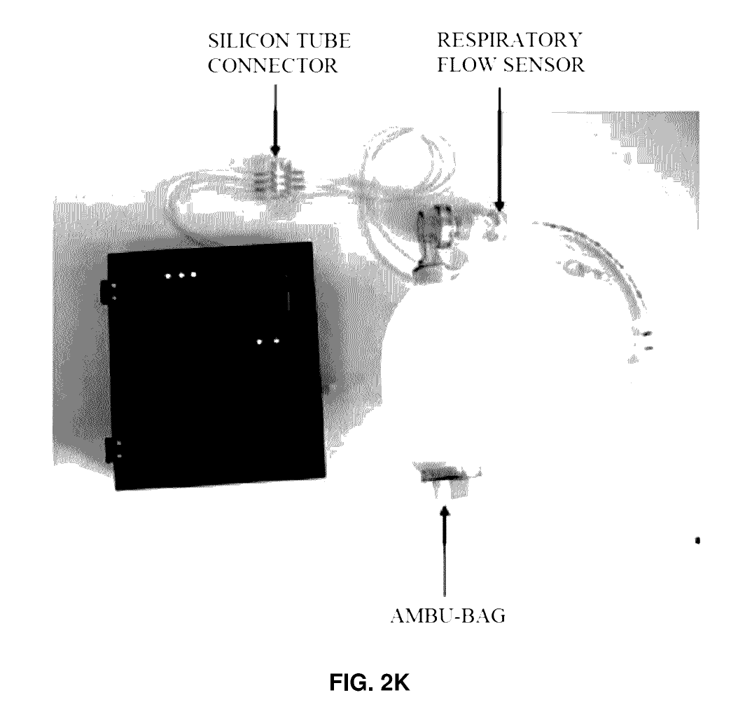

The preferred embodiments of the present invention will now be described with reference to the accompanying drawings. FIGS. 2A to 2E are diagrams illustrating a respiratory monitoring system according to an embodiment of the invention. FIG. 2F is a diagram illustrating a zero-point correction unit according to an embodiment of the invention. FIG. 2G is a graph illustrating a respiratory signal according to an embodiment of the invention. FIGS. 2H and 2I are diagrams illustrating a display unit according to an embodiment of the invention. FIG. 2J is a diagram illustrating another exemplary structure of first and second sensing tubes according to an embodiment of the invention. FIG. 2K is a photograph of the respiratory monitoring system according to an embodiment of the invention.

Referring to FIGS. 2A to 2D, the respiratory monitoring system according to an embodiment of the invention includes a first sensing tube 211, a second sensing tube 212, a third sensing tube 213, a filter 230, a first sensing element 221, a second sensing element 222, a third sensing element 223, a fourth sensing element 224, a signal extraction electronic circuit 240 a computation unit 250, a zero-point correction unit 280, a display unit 270, and a memory unit 260. The entire structure of the respiratory monitoring system according to an embodiment of the invention may be configured as illustrated in FIG. 2K. Referring to FIG. 2K, the first sensing tube 211, the second sensing tube 212, the third sensing tube 213, the first sensing element 221, the second sensing element 222, and the third sensing element 223 may constitute a respiratory flow sensor.

The first and second sensing tubes 211 and 212 is provided in a respiratory flow tube as a flow passage between an endo-tube and an ambu-bag of a breathing machine and has first and second directional holes opened depending on the respective respiratory flow direction. Specifically, the first and second sensing tubes 211 and 212 are cylindrical tubes having a diameter equal to or smaller than ⅓ of that of the respiratory flow tube. The first and second sensing tubes 211 and 212 are installed perpendicularly to a flow direction of the respiratory flow tube and have a plurality of first and second directional holes opened to face the corresponding respiratory flow.

For example, the first and second sensing tubes 211 and 212 are obtained by perforating first and second directional holes to a stainless cylindrical tube having an outer diameter of 1 mm and an inner diameter of 0.5 mm. The first and second sensing tubes 211 and 212 have a length exceeding the diameter of the respiratory flow passage and may be fixed to the respiratory flow tube not to vibrate (refer to FIGS. 2C and 2D). Note that the first and second directional holes may be opened oppositely to each other in parallel with the respiratory flow direction.

In this case, the closed ends of the first and second sensing tubes 211 and 212 may be fixed to an inner wall of the respiratory flow tube, and the opened ends thereof may be connected to the first and second sensing elements 221 and 222 through an outer wall of the respiratory flow tube. Note that one-side ends of the first and second silicon tubes are connected to the other-side ends of the first and second sensing tubes 211 and 212, respectively, and the other-side ends of the first and second silicon tubes are bisected and connected to the first and second sensing elements 221 and 222, respectively (refer to FIG. 2E). Typically, the flow velocity increases in the center of the respiratory flow tube, and the flow velocity decreases in the vicinity of the inner wall of the respiratory flow tube. According to the present invention, dynamic pressures at each representative points are physically averaged by perforating a plurality of sensing holes to the cylindrical flow sensing tube and connecting each pitot tube to each other just like a single pitot tube. On the basis of this strategy, it is possible to improve accuracy in the flow rate measurement. Furthermore, according to the present invention, the first and second cylindrical sensing tubes 211 and 212 have an outer diameter of approximately 1 mm so that they occupy a significantly small area in the cross-sectional area perpendicular to the respiratory flow tube. Therefore, it is possible to minimize a variation in the measurement characteristic caused by secretions discharged from an urgent critical patient from time to time.

The third sensing tube 213 is to measure an internal pressure of the respiratory flow tube. The third sensing tube 213 has one opened end and is installed in the respiratory flow tube through the wall of the respiratory flow tube. The other end of the third sensing tube 213 is opened and is connected to the third sensing element 223.

The filter 230 is installed in a chamber provided between the endo-tube of the breathing machine and the first to third sensing tubes 211 to 213 and filtrates secretions such as saliva or bloody phlegm from the endo-tube inserted into a patient's respiratory tract in order to prevent contamination of the first to third sensing tubes 211 to 213.

The first sensing element 221 detects a first dynamic pressure using a differential pressure between the gas flows from the first and second sensing tubes 211 and 212. In this case, the first sensing element 221 may be a differential pressure sensor having a sensitivity higher than that of the second sensing element 222 capable of measuring a pressure corresponding to a general artificial respiration range of 0 to ±2 L/sec.

The second sensing element 222 detects a second dynamic pressure using a differential pressure between the gas flows from the first and second sensing tubes 211 and 212. In this case, the second sensing element 222 may be a differential pressure sensor having a sensitivity lower than that of the first sensing element 221 capable of measuring a pressure of a high flow rate of −3 to +4 L/sec corresponding to the artificial respiration range of an urgent critical patient. Note that the inspiratory flow is denoted by a positive sign (+), and the expiratory flow is denoted by a negative sign (−) considering a characteristic of the respiratory flow direction.

The third sensing element 223 detects an internal pressure of the respiratory flow tube using a gas flow from the third sensing tube 213. In this case, the third sensing element 223 may be a pressure sensor capable of measuring an internal pressure of the respiratory flow tube with respect to the atmospheric pressure.

The fourth sensing element 224 is installed in the vicinity of the endo-tube of the breathing machine to measure a carbon dioxide concentration.

The signal extraction electronic circuit 240 is connected to each output of the first to third sensing elements 221 to 223 and has first to third amplifiers 241 to 243 and an analog-digital (A/D) converter 245.

The first amplifier 241 receives a first electric signal corresponding to the first dynamic pressure, amplifies the electric signal with a first gain, and outputs the amplified first electric signal. In this case, the first gain may be set to a value at which the first electric signal having a magnitude corresponding to a respiratory flow rate of 0.4 to 0.7 L/sec of a patient who can make a weak spontaneous respiration at the event of artificial respiration can be transformed to a voltage level of the computation unit 250.

The second amplifier 242 receives a second electric signal corresponding to the second dynamic pressure, amplifies the second electric signal with a second gain, and outputs the amplified second electric signal. In this case, the second gain may be set to a value at which a signal having a magnitude corresponding to a flow rate of −3 to 4 L/sec that may be instantaneously provided to an urgent critical patient can be transformed to a voltage level of the computation unit 250.

The third amplifier 243 receives a third electric signal corresponding to the internal pressure of the respiratory flow tube, amplifies the third electric signal with a third gain, and outputs the amplified third electric signal. In this case, the third gain may be set to a value at which the amplified third electric signal corresponding to the internal pressure of the respiratory flow tube can be transformed to a voltage level that can be detected by the computation unit 250.

The A/D converter 245 receives each output of the first to third amplifiers 241 to 243 and converts each received values into digital levels of the computation unit 250. The A/D converter 245 may be embedded in the computation unit 250. In this case, the A/D converter 245 may be omitted.

The computation unit 250 receives each output of the first to third amplifiers 241 to 243 or the digital values obtained by converting the output values of the first to third amplifiers 241 to 243 and computes patient's respiration information including a tidal inspiratory volume and a tidal expiratory volume.

The computation unit 250 may include at least one processing unit. For example, the processing unit may be a central processing unit (CPU), a graphic processing unit (GPU), an application specific integrated circuit (ASIC), a field programmable gate array (FPGA), or the like. The computation unit 250 may be provided with a plurality of cores.

The computation unit 250 computes a respiratory period including the inspiratory period and the expiratory period. Then, tidal inspiratory and expiratory volumes V_{I} and V_{E} can be computed by applying a simple mensuration-by-parts method to the flow rates of the inspiratory and expiratory periods as expressed in Formula 3. Here, “Tₛ” denotes a sampling interval of the flow rate and may be set to, for example, 0.01 [sec].

V=Ts·ΣF [Formula 3]

In this case, the computation unit 250 may compute the tidal inspiratory volume V_{I} by summing absolute values of the higher and lower flow rates used in computation of the inspiratory volume for the inspiratory period, multiplying the sum by a sampling interval, and converting the multiplication result into a milliliter scale [mL] as expressed in the following Formula 4 (refer to FIG. 2G).

$${V}_{I} = + {T}_{S} \times {\sum }_{{T}_{SI}}^{{T}_{EI}} \vert {F}_{H , L} \vert \qquad [ Formula 4 ]$$

In addition, the computation unit 250 may compute the tidal expiratory volume V_{E} by summing absolute values of the higher and lower flow rates used in computation of the expiratory volume for the expiratory period, multiplying the sum by a sampling interval, and converting the multiplication result into a milliliter scale [mL] as expressed in the following Formula 5 (refer to FIG. 2G).

$${V}_{E} = - {T}_{S} \times {\sum }_{{T}_{SE}}^{{T}_{EE}} \vert {F}_{H , L} \vert \qquad [ Formula 5 ]$$

In this case, the patient's respiration information contains at least one of a maximum value P_{T}MAX of the internal pressure of the respiratory tract for the inspiratory period (t=T_{SI} to T_{EI}), a maximum flow rate FMAX for the inspiratory period, an inspiratory time T_{I} [sec], an expiratory time T_{E} [sec], a ratio VRATIO between the tidal expiratory and inspiratory volumes, a respiration number per minute BPM [breaths/minute], a ratio Etol between the expiration and inspiratory periods, a respiratory period T_{E}+T_{I} [sec], a carbon dioxide concentration [%] at the end of expiration, and an operational status. Here, the operational status includes information on the operational status of the computation unit 250 during a zero point correction, an operation, or a boot-up procedure.

Note that the computation unit 250 may compute the inspiratory time T_{I} by calculating “T_{SE}−T_{SI}” and may compute the expiratory time T_{E} by calculating “T_{EE}−T_{SE}”.

Furthermore, the computation unit 250 obtains the maximum flow rate for the inspiratory period by selecting a maximum value out of the higher and lower flow rates for the inspiratory period as expressed in the following Formula 6.

FMAX=Max(F_{H,L}) [Formula 6]

The computation unit 250 can obtain a maximum value of the internal pressure of the respiratory tract for the inspiratory period using the internal pressure of the respiratory flow tube from the third sensing element 223. In addition, a carbon dioxide concentration at the end of the expiration can be obtained using the carbon dioxide concentration sensed by the fourth sensing element 224.

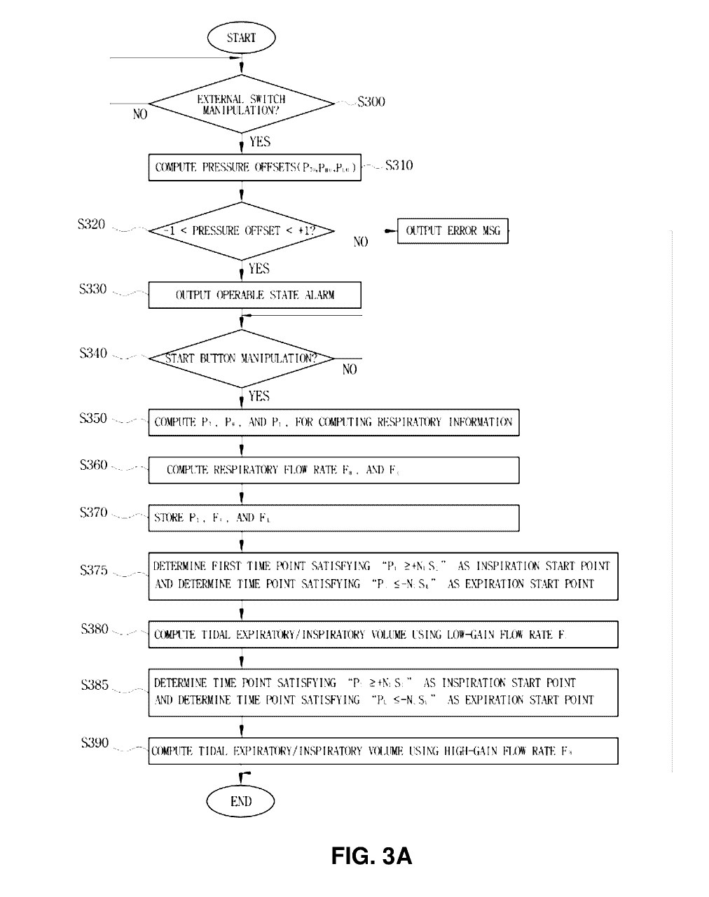

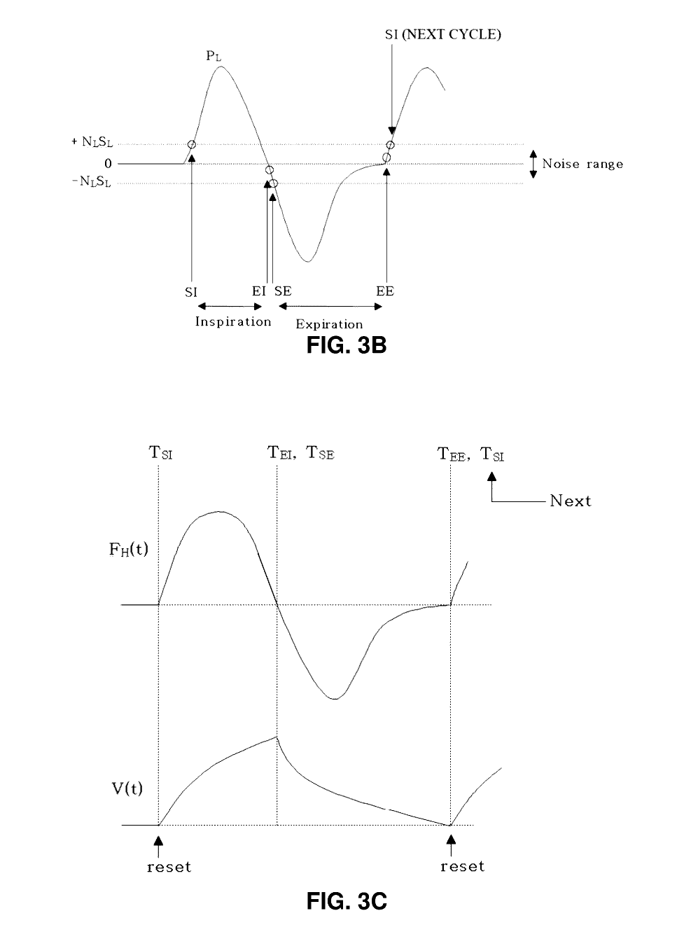

The process of computing the patient's respiration information in the computation unit 250 will be described below more specifically with reference to FIG. 3A.

The zero-point correction unit 280 is means for removing offset pressures of the first to third sensing elements 221 to 223. Specifically, the zero-point correction unit 280 has an external switch 283 and first and second open/close portions 281 and 282. The first open/close portion 281 is manipulated when the external switch 283 is manipulated, so that the first and second sensing tubes 211 and 212 are connected to each other. When the second open/close portion 282 is manipulated, the third sensing tube 212 is connected to the atmospheric pressure.

Note that the first and second open/close portions 281 and 282 may be opened during zero point correction and may be closed in other cases. For example, the first and second open/close portions 281 and 282 may be configured such that the silicon tube is closed or opened when the external switch 283 is manipulated as illustrated in FIG. 2F.

Note that the computation unit 250 can compute the pressure offsets for the lower flow pressure F_{L}, the higher flow pressure P_{H}, and the internal pressure P_{T} of the respiratory tract using the internal pressure of the respiratory flow tube and the first and second dynamic pressures detected when the first and second open/close portions 281 and 282 are opened by the zero-point correction unit 280.

The display unit 270 may display at least one type of the patient's respiration information in response to an instruction of the computation unit 250 as illustrated in FIG. 2H or 2I.

The memory unit 260 stores the patient's respiration information computed by the computation unit 250. The memory unit 260 may store the patient's respiration information on a time basis in a restorable format.

For example, the memory unit 260 may include a volatile memory (such as a random-access memory (RAM)), a non-volatile memory (such as a read-only memory (ROM) and a flash memory), or a combination thereof.

Meanwhile, in the embodiment described above, the first and second cylindrical sensing tubes 211 and 212 perpendicular to the respiratory flow tube have been exemplified. However, the first and second sensing tubes 211 and 212 may have any other shape. For example, the first and second sensing tubes 211 and 212 may have a cross shape as illustrated in FIG. 2J so that the flow rate of the horizontal direction as well as the flow rate of the vertical direction can be averaged.

Specifically, each sensing tube may be formed by connecting first and second cylindrical tubes having passages connected to each other in a cross shape. Both closed ends of the first cylindrical tube are fixed to the inner wall of the respiratory flow tube, and one opened end of the second cylindrical tube is fixed to the inner wall of the respiratory flow tube. The other end thereof may penetrate through the outer wall of the respiratory flow tube and may be connected to the first and second sensing elements 211 and 212.

In this manner, the system according to an embodiment of the invention may be applied as a small-sized patient's respiration information monitoring unit having a smart phone size to a manual type breathing machine usually employed before a patient transfer to a hospital or when a patient's position is changed in a hospital. As a result, the system according to an embodiment of the invention may be helpful to monitoring of an urgent critical patient.

According to an embodiment of the invention, a flow rate change inside a breathing machine is measured using a pair of pressure sensing elements having different sensitivities to differentiate the flow measurement range into two categories. As a result, it is possible to measure a maximum respiratory flow range that may be generated instantly as well as a general respiratory flow range. In addition, in a low flow range, it is possible to improve measurement accuracy using a high-sensitive pressure sensor. Accordingly, according to an embodiment of the invention, it is possible to support continuous monitoring of respiration information for an urgent cardiopulmonary arrest patient who have a respiratory flow change of 3 L/sec at maximum as well as a patient who can make a weak spontaneous respiration of 1.5 L/sec or lower.

According to an embodiment of the invention, it is possible to provide parameters such as a maximum internal pressure of the respiratory tract for the inspiratory period or a carbon dioxide concentration at the end of expiration. In addition, it is possible to prevent a rescuer from excessively pumping the ambu-bag as high as patient's pulmonary alveoli are damaged. Furthermore, it is possible to effectively operate a breathing machine in consideration of a patient's respiratory status.

According to an embodiment of the invention, it is possible to accurately analyze a patient's status by accumulating respiratory signals for a long period of time. Furthermore, it is possible to support establishment of database based on the analysis result or establishment of a guideline in consideration of various patient's conditions.

A respiratory monitoring method according to an embodiment of the invention will now be described with reference to FIGS. 3A to 3C. FIG. 3A is a flowchart illustrating a respiratory monitoring method according to an embodiment of the invention. FIGS. 3B and 3C are graphs illustrating respiratory signals according to an embodiment of the invention.

Referring to FIG. 3A, when it is detected that the external switch 283 is manipulated for a certain period of time (for example, 1 second) in step S300, the computation unit 250 computes the pressure offsets (that is, zero points P_{T0}, P_{H0}, and P_{L0}) for the lower flow pressure P_{L}, the higher flow pressure P_{H}, and the internal respiratory tract pressure P_{T} in step S310. Specifically, the computation unit 250 computes average values P_{T0}, P_{H0}, and P_{L0} of the endo-tube pressure and the first and second dynamic pressures as the pressure offsets. In addition, the computation unit 250 computes a standard deviation S_{L} of the average value P_{L0} of the second dynamic pressure and sets N_{L} times of the S_{L} value as a threshold. Here, “N_{L}” may be set to “5.”

The computation unit 250 checks whether or not the computed pressure offsets are allowable in step S320. For example, the computation unit 250 may determine that the pressure offsets are allowable if any one of the computed pressure offsets does not exceed a preset threshold range (for example, ±1 [cmH₂O]).

If the computed pressure offsets are allowable, the computation unit 250 outputs an alarm sound for notifying an operable state in step S330.

As a user presses a start button (YES in step S340), the computation unit 250 starts accumulation of the signals P_{T}, P_{H}, and P_{L} so that the signal values P_{T}, P_{H}, and P_{L} for computing respiration information are computed using the pressure offsets P_{T0}, P_{H0}, and P_{L0} in step S350. Specifically, the computation unit 250 may regard values obtained by subtracting the pressure offsets P_{T0}, P_{H0}, and P_{L0} from the accumulated values P_{T1}, P_{H1}, and P_{L1} as the signal values P_{T}, P_{H}, and P_{L} for computing respiration information.

The computation unit 250 computes the flow rates F_{H} and F_{L} using the signal values P_{H} and P_{L} for computing respiration information in step S360.

The computation unit 250 accumulates the computed internal endo-tube pressure P_{T} and the flow rates F_{H} and F_{L} in the memory unit 260 in step S370.

As illustrated in FIG. 3B, in step S375, the computation unit 250 detects an initial time point at which a condition “P_{L}≧+N_{L}S_{L}” is satisfied and determines it as an inspiration start point SI. In addition, the time of this moment is set as an inspiratory period start time T_{SI}. Furthermore, the computation unit 250 detects an initial time point at which a condition “P_{L}≦−N_{L}S_{L}” is satisfied and determines it as an expiration start point SE. In addition, the time of this moment is set as an expiratory period start time T_{SE}. Furthermore, the time point immediately before the expiration start point SE is set as inspiration end point EI (or T_{EI}).

In this case, at the first cycle after computation of the zero point, the computation unit 250 computes the tidal inspiratory volume by integrating the lower flow rate F_{L} for the inspiratory period and computes the tidal expiratory volume by integrating the lower flow rate F_{L} of the expiratory period in step S380.

Then, in step S385, the computation unit 250 detects a time point SI at which the condition “P_{L}≧+N_{L}S_{L}” is satisfied again (in the next cycle) and determines it as an inspiration start point of the next respiratory period. In addition, the time point immediately before this moment is set as an expiration end EE (or T_{EE}). The process is repeated. In this case, the computation unit 250 may reset the tidal inspiratory volume computed in the first cycle and the formula V(t) for computing the tidal expiratory volume. In this case, the computation unit 250 may accumulate the tidal inspiratory volume and the tidal expiratory volume of the previous cycle in the memory unit 260 as necessary. As illustrated in FIG. 3C, the computation unit 250 computes the tidal inspiratory volume and the tidal expiratory volume using the lower flow rate F_{L} at the second and subsequent cycles after computation of the zero point if the computed flow rate does not exceed a preset threshold value. In addition, the computation unit 250 computes the tidal inspiratory volume and the tidal expiratory volume by partially applying the higher flow rate F_{H} to the flow rate exceeding the threshold value.

Meanwhile, if it is determined in step S320 that at least one of the computed pressure offsets is not allowable, the computation unit 250 may output an error message in step S390.

If it is detected that a user presses an END button in the processes described above, the computation unit 250 may interrupt accumulation of the respiratory signals. If it is detected that the external switch 283 is manipulated in the middle of signal accumulation, the process may return to step S310. The start button and the end button described above may be provided separately from the external switch 283. Alternatively, the external switch 283, the start button, and the end button may be classified depending on the number of manipulation.

In this manner, according to the present invention, it is possible to detect a respiratory period including inspiration (T_{SI} to T_{EI}) and expiration (T_{SE} to T_{EE}) by repeatedly detecting the inspiration and expiration start points.

In this case, a principle of the respiratory period computation is similar to the principle of the Schmitt trigger circuit. Therefore, it is impossible that the respiratory period has solely an inspiratory period or an expiratory period.

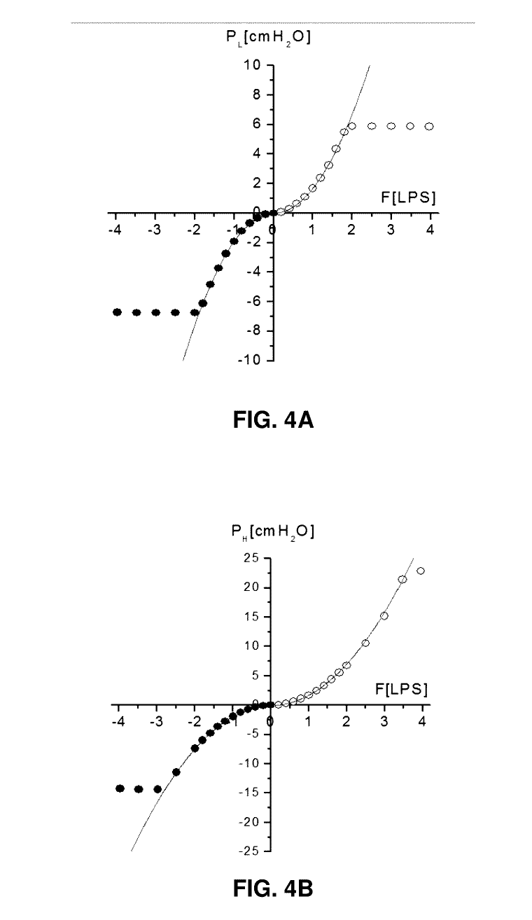

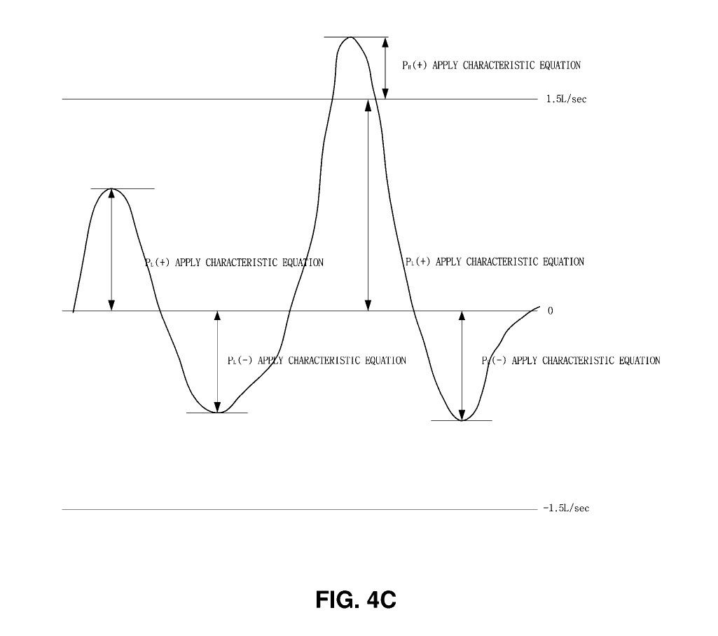

Flow rate computation accuracy of the respiratory monitoring system according to an embodiment of the invention will now be described with reference to FIGS. 4A to 4C. FIG. 4A is a graph illustrating a correlation between the first dynamic pressure and the flow rate according to an embodiment of the invention. FIG. 4B is a graph illustrating a correlation between the second dynamic pressure and the flow rate according to an embodiment of the invention. FIG. 4C is a graph obtained by using an exemplary characteristic expression of the respiratory signal according to an embodiment of the invention.

First, an experimental method for measuring the correlation between the dynamic pressure and the flow rate will be described in brief. In this experiment, a standard connector and an endo-tube are connected sequentially to the left side of the respiratory flow sensor, and a standard flow generator instead of the ambu-bag is connected to the right side in order to enable a quantitative respiratory flow.

Note that the standard flow generator has a cylindrical main body having a constant inner diameter and a servomotor (for example, model No. CSDJ-10BX2, produced by Samsung Electronics Co. Ltd., South Korea) driven to generate any constant gas flow. In addition, a linear displacement sensor (For example, model No. LTM600S, produced by Gefran, Italy) is connected to a driving shaft of the servomotor so that a position (volume V) signal depending on a syringe movement is output continuously. As a result, it is possible to accurately measure the amount of the gas passing through the sensor. In addition, when the piston of the standard flow generator moves from the right to the left, the gas is discharged through the respiratory gas flow sensor and the endo-tube to simulate an inspiratory state of real respiration. In contrast, when the syringe moves from the right to the left, an expiration state is reflected.

In this case, while the flow is maintained constantly, the volume V is changed linearly. Therefore, the gradients F of the volume V for an interval in which the volume V is constantly increased or decreased were computed, and they are plotted along the x-axis in FIGS. 4A and 4B. In FIGS. 4A and 4B, the y-axis denotes an average of the first and second dynamic pressures generated in the same interval as that used in the computation of the gradient F (refer to the red line in FIGS. 4A and 4B).

For the interval in which the outputs of the first and second sensing elements are increased or decreased constantly, a correlation between the first dynamic pressure and the gradient F and a correlation between the second dynamic pressure and the gradient F were computed through a quadratic function fitting. As a result, a correlation coefficient was 0.999 or greater. Characteristic expressions computed based thereupon were obtained as expressed in Formulas 7 and 8 and the red lines of FIGS. 4A and 4B.

P_{L}(+)=1.68F²+0.01F

P_{L}(−)=−1.88F²+0.03F [Formula 7]

P_{H}(+)=1.80F²−0.09F,

P_{H}(−)=−1.81F²+0.11F [Formula 8]

If the first and second flow rates (indicated by the circles in FIGS. 4A and 4B) computed by the computation unit 250 from the first and second dynamic pressures are compared with the pressures computed from the Formulas 7 and 8 described above (red lines in FIGS. 4A and 4B), it is recognized that the flow rate corresponding to the first dynamic pressure is saturated approximately at “2 L/sec,” and the flow rate corresponding to the second dynamic pressure is saturated approximately at “3.6 L/sec.”

Therefore, if the computed pressure is lower than 1.5 L/sec, the computation unit 250 according to an embodiment of the invention computes the respiratory flow rate by applying the first dynamic pressure to Formula 7. In contrast, if the computed pressure exceeds 1.5 L/sec, that may be generated in emergency, the respiratory flow rate can be computed by applying the first dynamic pressure to Formula 7 for a flow rate of 1.5 L/sec or lower and applying the second dynamic pressure to Formula 8 for a flow rate exceeding 1.5 L/sec (refer to FIG. 4C). Therefore, it is possible to compute the respiratory flow up to a high flow rate range using conditional formula application. As a result, according to the present invention, it is possible to easily compute the respiratory flow rate across the entire range of the artificial respiratory flow.

The standard respiration information and the respiration information measured using Formulas 4 and 5 were compared for inspiratory and expiratory periods according to an embodiment of the invention. As a result, as shown in the following Table 1, it is recognized that very accurate measurement can be performed within a mean relative error of 3%. Note that the resultant values of Table 1 were obtained under an exemplary experimental environment, and the measurement values may be changed depending on the experimental environment.

TABLE 1

| | Inspiratory | Expiratory | | Inspiratory | Expiratory | | |
|---|---|---|---|---|---|---|---|
| Designed | Volume | Volume | Standard | Volume | Volume | Inspiration | Expiration |
| Waveform | (L) | (L) | Waveform | (L) | (L) | % e | % e |
| #1 | 1.590 | −1.149 | +1 | 1.600 | −1.181 | −0.625 | −2.710 |
| #2 | 1.621 | −1.275 | +2 | 1.597 | −1.278 | 1.503 | −0.235 |
| #3 | 1.538 | −0.978 | +3 | 1.475 | −1.070 | 4.271 | −8.598 |
| #4 | 1.504 | −1.466 | +4 | 1.530 | −1.460 | 1.699 | 0.411 |
| #5 | 1.582 | −1.297 | +5 | 1.592 | −1.314 | −0.628 | −1.294 |
| #6 | 1.496 | −1.282 | +6 | 1.524 | −1.296 | −1.837 | −1.080 |

| | | |
|---|---|---|
| Mean (l % el) | 1.761 | 2.388 |
| SD | 1.338 | 3.166 |

In this manner, according to an embodiment of the invention, it is possible to easily provide patient's respiration information with high accuracy using characteristic equations.

While preferred embodiments of the invention have been described and illustrated hereinbefore, it should be understood that they are only for exemplary purposes and are not to be construed as limiting. Any addition, omission, substitution, or modification may be possible without departing from the spirit or scope of the present invention. Accordingly, the invention is not to be considered as being limited by the foregoing description, and is only limited by the scope of the appended claims.

What is claimed is:

1. A respiratory monitoring system comprising:
a first sensing tube provided in a respiratory flow tube serving as a flow passage of a breathing machine and provided with at least a first directional hole opened in a respiratory flow direction;
a second sensing tube provided with at least a second directional hole corresponding to the first directional hole and provided in the vicinity of the first sensing tube;
a first sensing element configured to detect a first dynamic pressure (F_{L}) using a differential pressure between gas flows from the first and second sensing tubes;
a second sensing element configured to detect a second dynamic pressure (P_{H}) using a differential pressure between gas flows from the first and second sensing tubes, the second sensing element having sensitivity lower than that of the first sensing element and a sensing range wider than that of the first sensing element; and
a computation unit configured to compute patient's respiration information including a tidal inspiratory volume and a tidal expiratory volume using the first and second dynamic pressures,
wherein the computation unit computes the respiration information using a lower flow rate (F_{L}) if the lower flow rate (F_{L}) computed from the first dynamic pressure is smaller than a preset threshold value, and
the computation unit computes the respiration information using a higher flow rate (F_{H}) computed from the second dynamic pressure if the lower flow rate (F_{L}) is greater than the threshold value.

2. The respiratory monitoring system according to claim 1, further comprising:
a first amplifier configured to amplify a first electric signal corresponding to the first dynamic pressure with a first gain and provide the amplified first electric signal to the computation unit; and
a second amplifier configured to amplify a second electric signal corresponding to the second dynamic pressure with a second gain and provide the amplified second electric signal to the computation unit,
wherein the computation unit computes the patient's respiration information using an output of the first amplifier or an output of the second amplifier.

3. The respiratory monitoring system according to claim 1, wherein the first and second sensing tubes are cylindrical tubes installed perpendicularly to the flow direction between an endo-tube and an ambu-bag of the breathing machine, and
one-side ends of the first and second sensing tubes are fixed to an inner wall of the respiratory flow tube, and the other-side ends thereof are connected to the first and second sensing elements through an outer wall of the respiratory flow tube.

4. The respiratory monitoring system according to claim 1, wherein the first and second sensing tubes are formed by bonding first and second cylindrical tubes having passages connected to each other in a cross shape,
both closed ends of the first cylindrical tube are fixed to an inner wall of the respiratory flow tube,
one opened end of the second cylindrical tube is fixed to the inner wall of the respiratory flow tube, and
the other opened end of the second cylindrical tube is connected to the first and second sensing elements through an outer wall of the respiratory flow tube.

5. The respiratory monitoring system according to claim 1, further comprising a fourth sensing element that detects a carbon dioxide concentration in the respiratory flow tube,
wherein the computation unit computes a carbon dioxide concentration at the end of expiration included in the respiration information using information detected by the fourth sensing element.

6. The respiratory monitoring system according to claim 1, further comprising a third sensing element provided in the respiratory flow tube to measure an internal pressure of the respiratory flow tube,
wherein the computation unit computes a maximum respiratory tract internal pressure for an expiratory period included in the respiration information using the internal pressure of the respiratory flow tube.

7. The respiratory monitoring system according to claim 1, further comprising an open/close portion for connecting or disconnecting the first and second sensing tubes,
wherein the computation unit computes a high-gain pressure offset and a low-gain pressure offset using first and second dynamic pressures detected when the first and second sensing tubes are connected using the open/close portion, and
the computation unit computes the patient's respiration information using a result of correcting the lower flow rate and the higher flow rate on the basis of the high-gain pressure offset and the low-gain pressure offset.

8. The respiratory monitoring system according to claim 1, wherein the computation unit
determines a time point at which the lower flow rate is equal to or higher than a positive (+) value of a value obtained by multiplying a zero-point average value (S_{L}) of the first dynamic pressure for a certain period of time by a factor “N_{L}” (where “N_{L}” denotes a natural number equal to or greater than “1”) as an inspiration start point,
determines a time point at which the lower flow rate is equal to or lower than “−S_{L}×N_{L}” as an expiration start point, and
computes the respiration information including the tidal expiratory volume and the tidal inspiratory volume using the lower flow rate regardless a result of comparison between the higher flow rate and the threshold value in a respiratory period started at the first inspiration start point after computation of the zero-point average value.

9. The respiratory monitoring system according to claim 1, further comprising a display unit configured to display the respiration information.

10. The respiratory monitoring system according to claim 1, wherein the respiration information includes at least one of a maximum respiratory tract internal pressure (P_{T}MAX) during an inspiratory period (t=T_{SI} to T_{EI}), a maximum flow rate (FMAX) during an inspiratory period, an inspiration time (T_{I}), an expiration time (T_{E}), a ratio (VRATIO) between a tidal expiratory volume and a tidal inspiratory volume, a breathing number per minute (BPM), a ratio (Etol) between the expiratory period and the inspiratory period, a respiratory period (T_{E}+T_{I}), a carbon dioxide concentration [%] at the end of expiration, and an operational status of the computation unit.

11. The respiratory monitoring system according to claim 1, wherein the computation unit computes the tidal inspiratory volume by summing absolute values of the higher flow rates computed during the inspiratory period or a flow rate used in computation of respiration information out of the higher flow rates and multiplying the sum of the absolute values by a sampling interval, and
the computation unit computes the tidal expiratory volume by summing absolute values of the flow rates computed during the expiratory period and used in computation of the respiration information and multiplying the sum of the absolute values by a sampling interval.

12. A respiratory monitoring method using a respiratory monitoring system having
a first sensing tube provided in a respiratory flow tube serving as a flow passage of a manual breathing machine and provided with at least a first directional hole opened in a respiratory flow direction,
a second sensing tube provided with a second directional hole corresponding to the first direction hole and provided in the vicinity of the first sensing tube,
a first sensing element configured to detect a first dynamic pressure (P_{L}) using a differential pressure between gas flows from the first and second sensing tubes,
a second sensing element configured to detect a second dynamic pressure (P_{H}) using a differential pressure between gas flows from the first and second sensing tubes, the second sensing element having sensitivity lower than that of the first sensing element and a sensing range wider than that of the first sensing element, and
a computation unit,
the respiratory monitoring method comprising:
computing patient's respiration information including a tidal inspiratory volume and a tidal expiratory volume using the first and second dynamic pressures,
wherein the respiration information is computed using a lower flow rate (F_{L}) if the lower flow rate (F_{L}) computed from the first dynamic pressure is smaller than a preset threshold value, and
the respiration information is computed using a higher flow rate (F_{H}) computed from the second dynamic pressure if the lower flow rate (F_{L}) is greater than the threshold value.

13. The respiratory monitoring method according to claim 12, wherein the computing includes
determining a time point at which the lower flow rate is equal to or higher than a positive (+) value of a value obtained by multiplying a zero-point average (S_{L}) of the first dynamic pressure for a certain period of time by a factor “N_{L}” (where “N_{L}” denotes any natural number equal to or greater than “1”) as an inspiration start point,
determining a time point at which the lower flow rate is equal to or lower than “−S_{L}×N_{L}” as an expiration start point, and
computing the respiration information including the tidal expiratory volume and the tidal inspiratory volume using the lower flow rate regardless of a result of comparison between the higher flow rate and the threshold value in a respiratory period started at the first inspiration start point after computation of the zero-point average.
